(19) United States
(12) Patent Application Publication
(10) Pub. No.: US 2012/0288849 A1
(43) Pub. Date: Nov. 15, 2012

(54) METHOD AND SYSTEM FOR ROBUST AND SENSITIVE ANALYSIS OF BEAD-BASED ASSAYS

(75) Inventors: Joong-Ho Won, Cupertino, CA (US); Ofir Goldberger, Menlo Park, CA (US); Mark M. Davis, Atherton, CA (US); Richard A. Olshen, Stanford, CA (US)

(73) Assignee: The Board of Trustees of the Leland Stanford, Junior, University, Palo Alto, CA (US)

(21) Appl. No.: 13/364,302

(22) Filed: Feb. 1, 2012

Related U.S. Application Data
(60) Provisional application No. 61/465,239, filed on Mar. 15, 2011.

(51) Int. Cl. G01N 33/566; G06F 19/00; G01N 33/53

(52) U.S. Cl. 435/5; 436/501; 435/7.1; 435/7.4; 435/7.92; 435/7.93; 435/7.94; 702/22

(57) ABSTRACT

Computer-implemented methods and systems are provided for the analysis of multiplex fluorescent-dyed microsphere assays. The methods of the invention provide for determination of differences in analyte quantities between samples obtained from multiplex fluorescent-dyed microsphere assays by analysis of individual bead fluorescence and adjusting for variance; variance-stabilization of the data, and determining significance with hypothesis testing with tolerance determined by power estimation. The methods of the invention provide a benefit in allowing access to low signal or poor quality data, increased statistical power and decreased variability compared to standard curve methodology.

### FIELD OF THE INVENTION

The present invention generally relates to the field of biomedical informatics. More particularly, the present invention relates to methods for analyzing multiplex microsphere assays.

### BACKGROUND OF THE INVENTION

For many purposes relating to biological assays, simultaneous information is obtained for multiple parameters. Examples of such assays include analysis of nucleic acids, expression of proteins such as cytokines, and the like. For example, cytokine expression profiling is used for the identification and characterization of disease-associated immune responses.

Conventional methods for performing such multiplex analysis are fluorescent bead-based technologies, which typically combine a flow cytometer, fluorescent-dyed microspheres (xMap beads), lasers and digital signal processing. The xMap technology-based instrument and a general flow cytometer share basic technology such as lasers, fluidics, and optics. In addition, they both have the ability to identify and report the result of a microsphere-based assay. xMap technology uses a single 5.6 micron size microsphere and a proprietary dying process to create unique dye mixtures which are used to identify an individual microsphere.

The interpretation of these large numbers of data points requires statistical analysis. Currently, measured levels of fluorescence from the known analyte dilutions are used to compute median fluorescence intensities (MFIs) and create standard curves, which then allow estimation of unknown concentrations of analytes. Conventional standard curve based analysis methods have limitations in detecting low or high abundance analytes and also introduce significant error as a result of dilution variation, fluorescence readout error (machine error) and curve fitting error. Alternatively, the MFI values themselves can be used, but this does not solve sensitivity constraints or low statistical power issue with small sample numbers.

Current methods for statistical analysis of xMap assays rely on repeat wells done in the assay and point estimators, usually the concentrations transformed from the MFIs, for each analyte within each well. This approach may be adequate when a large difference exists and where CVs are relatively small. But in highly multiplex assays, one or more analytes are typically analyzed at non-optimal levels, and may generate very high or very low values, resulting in incorrect reporting of the relative concentrations. For example, this can happens when an MFI is computed to be below the intersection of the standard curve and the ordinate. These values lead to sparse results and also provide poor estimation of the levels of analytes. The present invention addresses these and other issues.

### SUMMARY OF THE INVENTION

Among other things, the present invention provides computer-implemented methods, storage mediums, and systems for performing one or more steps associated with data processing of multiplex fluorescent-dyed microsphere assays. Embodiments of the present invention provide for the determination of differences in analyte quantities between samples obtained from multiplex fluorescent-dyed microsphere assays by direct statistical analysis of fluorescence intensities of individual beads and adjusting for intrasample variance, as opposed to analysis based on a summary number. The methods of the present invention provide a benefit in allowing access to low signal or poor quality data; and more power to testing differences in analytes.

Embodiments of the computer-implemented methods, storage media, and systems are configured to provide Statistical Analysis of xMap Cytokine Beads (SAxCyB). Embodiments for statistical analysis are adapted for use with the heavy-tailed distribution and the high variability typical of the data obtained from xMap beads.

Systems using xMap technology perform a variety of multiplexed bead assays, including immunoassays, on the surface of fluorescent-coded beads, which are then read in a compact analyzer. Using two lasers and high-speed digital-signal processors, the analyzer reads signals on each individual microsphere particle. The capability of adding multiple conjugated beads to each sample results in the ability to obtain multiple data points from each sample. The statistical analysis methods of embodiments of the present invention as described herein provide a means for determining differences in multiple analyte from such data, among other things.

In some embodiments, a method is provided for determining the difference in distribution of an analyte of interest, usually relative to a control sample, where a sample suspected of comprising one or more analyte(s) of interest is contacted with (i) detectably-labeled reporters comprising a binding member specific for the analyte of interest and (ii) xMap beads having (a) a binding member specific for an analyte of interest and (b) an embedded unique identifier (such as a two-color barcode). Typically a plurality of such xMap beads and detectably-labeled reporters are multiplexed in an assay with each sample, however this is not required. In certain cases, the detectable label is a fluorescent label. The sample is subjected to excitation, and the resulting emission of the barcode and the reporter is recorded. Replicates of each sample are analyzed, e.g. two, three or more replicates. Each xMap bead is taken to be a single data point comprising doublet discriminator values and doublet discriminator decision (e.g., true or false), barcode classification data and reporter emission data. Data are subjected to an algorithm according to an embodiment of the present invention in order to find differences between quantities of multiple analytes.

In the linear regression, data from replicates of each sample are combined after adjusting for differences. Samples of interest are simultaneously compared to reference conditions after taking variance-stabilizing transform. In an embodiment, the transform uses a “blank” with no analytes. Comparisons are made through a bioequivalence-type hypothesis testing, in which the equivalence margin is determined using a data-driven statistical power estimation procedure.

The following are mere exemplary embodiments of the computer-implemented methods, storage mediums, and systems and are not to be construed in any way to limit the subject matter of the claims. These and other embodiments can be more fully appreciated upon an understanding of the detailed description of the invention as disclosed below in conjunction with the attached figures.

### BRIEF DESCRIPTION OF THE DRAWINGS

The following drawings will be used to more fully describe embodiments of the present invention.

FIG. 1a is a schematic flow chart illustrating a method according to an embodiment of the present invention.

FIG. 1b are illustrations of various applications of a method according to an embodiment of the present invention for experiments with different variances.

FIG. 2a includes certain graphs that demonstrate the performance of SAxCyB as an embodiment of the present invention.

FIG. 2b includes certain graphs that illustrate the use of SAxCyB as an embodiment of the present invention in analysis of a cytokine stimulation assay.

FIG. 3 includes quantile-quantile plots of typical xMap bead fluorescence intensity (FI) data for 21 standard analytes. The shown straight lines in the various graphs represent the standard normal distribution from which it is observed that the collected data varies.

FIG. 4 includes an exemplary scale of xMap bead fluorescence intensity data for eotaxin as observed in an application of the present invention.

FIG. 5 is a graph that illustrates the tradeoffs associated with choosing an appropriate equivalence margin (Δ) using the power estimated from the data.

FIG. 6 is a graph of FPR versus TPR in an application of an embodiment of the present invention.

FIG. 7 is a block diagram of a computer system on which the present invention can be implemented.

FIG. 8 is a flowchart of a method according to an embodiment of the present invention.

FIG. 9 is an illustration of a transformation according to an embodiment of the present invention.

### DETAILED DESCRIPTION OF THE INVENTION

Among other things, the present invention relates to methods, techniques, and algorithms that are intended to be implemented in a digital computer system 100 such as generally shown in FIG. 14. Such a digital computer is well-known in the art and may include the following.

Computer system 100 may include at least one central processing unit 102 but may include many processors or processing cores. Computer system 100 may further include memory 104 in different forms such as RAM, ROM, hard disk, optical drives, and removable drives that may further include drive controllers and other hardware. Auxiliary storage 112 may also be include that can be similar to memory 104 but may be more remotely incorporated such as in a distributed computer system with distributed memory capabilities.

Computer system 100 may further include at least one output device 108 such as a display unit, video hardware, or other peripherals (e.g., printer). At least one input device 106 may also be included in computer system 100 that may include a pointing device (e.g., mouse), a text input device (e.g., keyboard), or touch screen.

Communications interfaces 114 also form an important aspect of computer system 100 especially where computer system 100 is deployed as a distributed computer system. Computer interfaces 114 may include LAN network adapters, WAN network adapters, wireless interfaces, Bluetooth interfaces, modems and other networking interfaces as currently available and as may be developed in the future.

Computer system 100 may further include other components 116 that may be generally available components as well as specially developed components for implementation of the present invention. Importantly, computer system 100 incorporates various data buses 116 that are intended to allow for communication of the various components of computer system 100. Data buses 116 include, for example, input/output buses and bus controllers.

Indeed, the present invention is not limited to computer system 100 as known at the time of the invention. Instead, the present invention is intended to be deployed in future computer systems with more advanced technology that can make use of all aspects of the present invention. It is expected that computer technology will continue to advance but one of ordinary skill in the art will be able to take the present disclosure and implement the described teachings on the more advanced computers or other digital devices such as mobile telephones or “smart” televisions as they become available. Moreover, the present invention may be implemented on one or more distributed computers. Still further, the present invention may be implemented in various types of software languages including C, C++, and others. Also, one of ordinary skill in the art is familiar with compiling software source code into executable software that may be stored in various forms and in various media (e.g., magnetic, optical, solid state, etc.). One of ordinary skill in the art is familiar with the use of computers and software languages and, with an understanding of the present disclosure, will be able to implement the present teachings for use on a wide variety of computers.

Before the present invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may be included independently in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned hereunder are incorporated herein by reference. Unless mentioned otherwise, the techniques employed herein are standard methodologies well known to one of ordinary skill in the art.

It must be noted that as used herein and in the appended claims, the singular forms “a,” “and,” and “the” include plural referents unless the context clearly dictates otherwise. Thus, for example, reference to “a biomarker” includes a plurality of such biomarkers and reference to “the sample” includes reference to one or more samples and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Moreover any positively recited element of the disclosure provides basis for a negative limitation to exclude that element from the claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Other objects, features and advantages of the present invention will become apparent from the following detailed description. It should be understood, however, that the detailed description and specific examples, while indicating preferred embodiments of the invention, are given by way of illustration only, since various changes and modifications within the spirit and scope of the invention will become apparent to those skilled in the art from this detailed description.

Unless otherwise indicated, the practice of the present invention will employ conventional techniques of molecular biology (including recombinant techniques), microbiology, cell biology, biochemistry and immunology, which are within the skill of the art. Such techniques are explained fully in the literature, such as, “Molecular Cloning: A Laboratory Manual,” second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction,” (Mullis et al., eds., 1994); and “Current Protocols in Immunology” (J. E. Coligan et al., eds., 1991).

As used throughout, “modulation” is meant to refer to an increase or a decrease in the indicated phenomenon (e.g., modulation of a biological activity refers to an increase in a biological activity or a decrease in a biological activity).

As used herein, the terms “determining,” “assessing,” “assaying,” “measuring,” and “detecting” refer to both quantitative and qualitative determinations and as such, the term “determining” is used interchangeably herein with “assaying,” “measuring,” and the like. Where a quantitative determination is intended, the phrase “determining an amount” and the like is used. Where either a qualitative or quantitative determination is intended, the phrase “determining a level of proliferation” or “detecting proliferation” is used.

Although embodiments are described herein with respect to particles, it is to be understood that the systems and methods described herein may also be used with microspheres, polystyrene beads, microparticles, gold nanoparticles, quantum dots, nanodots, nanoparticles, nanoshells, beads, microbeads, latex particles, latex beads, fluorescent beads, fluorescent particles, colored particles, colored beads, tissue, cells, micro-organisms, organic matter, non-organic matter, or any other discrete substances known in the art. The particles may serve as vehicles for molecular reactions.

Examples of appropriate particles are illustrated and described in U.S. Pat. No. 5,736,330 to Fulton, U.S. Pat. No. 5,981,180 to Chandler et al., U.S. Pat. No. 6,057,107 to Fulton, U.S. Pat. No. 6,268,222 to Chandler et al., U.S. Pat. No. 6,449,562 to Chandler et al., U.S. Pat. No. 6,514,295 to Chandler et al., U.S. Pat. No. 6,524,793 to Chandler et al., and U.S. Pat. No. 6,528,165 to Chandler, which are incorporated by reference as if fully set forth herein. The systems and methods described herein may be used with any of the particles described in these patents. In addition, particles for use in method and system embodiments described herein may be obtained from manufacturers such as Luminex Corporation of Austin, Tex. The terms “particles” and “microspheres” are used interchangeably herein.

In addition, the types of particles that are compatible with the systems and methods described herein include particles with fluorescent materials attached to, or associated with, the surface of the particles. These types of particles, in which fluorescent dyes or fluorescent particles are coupled directly to the surface of the particles in order to provide the classification fluorescence (i.e., fluorescence emission measured and used for determining an identity of a particle or the subset to which a particle belongs), are illustrated and described in U.S. Pat. No. 6,268,222 to Chandler et al. and U.S. Pat. No. 6,649,414 to Chandler et al., which are incorporated by reference as if fully set forth herein. The types of particles that can be used in the methods and systems described herein also include particles having one or more fluorochromes or fluorescent dyes incorporated into the core of the particles.

In an embodiment of the present invention, particles of this type may be analyzed by an instrument having a three-color fluorescence signal-detection system. Two colors are dedicated to microsphere classification; the third color is used for measurement of the reporter fluorescence intensity. Many other embodiments are possible as would be understood by one of ordinary skill in the art upon understanding the teachings of the present invention.

Particles that can be used in the methods and systems described herein further include particles that in of themselves will exhibit one or more fluorescent signals upon exposure to one or more appropriate light sources, or any other detection system (e.g., chemiluminescence). Furthermore, particles may be manufactured such that upon excitation the particles exhibit multiple fluorescent signals, each of which may be used separately or in combination to determine an identity of the particles. As described below, data processing may include a determination of the amount of analyte bound to the particles.

Analyte, as used herein is a broad term and is used in its ordinary sense as a substance the presence, absence, or quantity of which is to be determined, including, without limitation, to refer to a substance or chemical constituent in a fluid such as a biological fluid or cell or population of cells that can be analyzed. An analyte can be a substance for which a naturally occurring binding member exists, or for which a binding member can be prepared. Non-limiting examples of analytes include, for example, antibodies, antigens, polynucleotides, polypeptides, proteins, hormones, cytokines, growth factors, steroids, vitamins, toxins, drugs, and metabolites of the above substances, as well as bacteria, viruses, fungi, fungal spores and the like.

An “analyte-specific probe” as used herein, is a probe capable of specifically binding to the analyte and to which a label can be attached. The binding of the probe to the analyte can be based on any type of interaction including but not limited to complementary nucleotide sequences, antigen/antibody interaction, ligand/receptor binding, enzyme/substrate interaction, etc.

The term “test sample,” as used herein, refers to a sample that may contain the analyte of interest. For example, the test sample may be a culture medium, cell, cell lysate, biological fluid or tissue, such as whole blood or whole blood components (including red blood cells, white blood cells, platelets, serum and plasma), ascites, urine, cerebrospinal fluid, saliva, breath condensate, fluid obtained by lavage or other constituents of the body that may contain the analyte of interest.

As used herein, the term “antibody” includes monoclonal antibodies and monospecific polyclonal antibodies, and both intact molecules as well as antibody fragments (such as, Fab, Fab' and F(ab′)2, Fd, single-chain Fvs (scFv), single-chain antibodies, disulfide-linked Fvs (sdFv) and fragments comprising either a VL or VH domain) which are capable of specifically binding to a target analyte.

The term “label,” as used herein, refers to a molecule or material capable of generating a detectable signal. The term “fraction of bound label” as used herein refers to those labels, which, when adding a predetermined amount of label to a sample, bind to the analyte. The term “fraction of unbound label” as used herein refers to those labels which, when adding a predetermined amount of label to a sample, do not bind to the analyte.

A “capture probe” as used herein refers to a molecule capable of binding a molecule or a complex of molecules to a substrate.

A “substrate” as used herein refers to a material, to which molecules or complexes of molecules can be bound, and which can be manipulated. Typical examples of substrates include but are not limited to microtiter plates, beads, chips, etc.

The term fluorescence ELISA as used herein refers to an antibody-based assay in which detection of an analyte is accomplished via binding of a labeled antibody to an analyte producing a detectable signal. An ELISA can be run in competitive or non-competitive formats. ELISA also includes a 2-site or “sandwich” assay in which two antibodies to the antigen are used. In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase or horseradish peroxidase, both of which generate a detectable product when contacted by appropriate substrates.

The present invention provides methods and compositions relating to methods of analysis for data obtained from multiplex experiments, by determining the relative quantities of analyte bound to an xMap particle. Multiplex methods by necessity make compromises with respect to the dynamic range of certain analytes. The methods of the invention address the data analysis problem, negate the requirement for technical repeats, and provide powerful statistical testing due to the increase in sample size.

Assays are carried out in accordance with various protocols. A sample suspected of comprising one or more analyte(s) of interest is contacted with (i) detectably-labeled reporters comprising a binding member specific for the analyte of interest and (ii) xMap beads having (a) a binding member specific for an analyte of interest and (b) an embedded two-color barcode. Typically a plurality of such xMap beads and detectably-labeled reporters are multiplexed in an assay with each sample.

Various operations may be carried out, such as the addition of miscellaneous reagents, incubations, washings, and the like. Usually the detectable label is a fluorescent label. The sample is subjected to excitation. The final result of the assays is the change in the amount of a product, which absorbs or produces light, either by light absorption or by light emission in relation to the presence or amount of the analyte of interest. Usually, this is as a result of formation of a specific binding complex between complementary members of a specific binding pair, where one of the members may serve as a bridge to form a sandwich (as in “sandwich” assay), or there may be a single complex, or complexes may be bound to complex binding proteins, such as S. aureus protein A, rheumatoid factor, immunoglobulins specific for immune complexes, or the like.

In embodiments of the present invention, by having fluorescent markers, such as fluorescent particles, fluorescent conjugated antibodies, or the like, the sample may be irradiated with light absorbed by the fluorescers and the emitted light measured by light measuring devices. Dyes can be employed as the label or produced as a result of a reaction, e.g. an enzymatically catalyzed reaction.

In another embodiment of the present invention such as may be used with nucleic acid assays involving hybridization, one can carry out the necessary steps to determine whether complementary sequences are present, and by employing a wide variety of protocols known to those of ordinary skill in the art, provide for a colored or fluorescent label or product of the label, which will indicate the presence or absence of the complementary sequence.

In yet another embodiment of the present invention that uses biological or chemical modifications (e.g., phosphorylation, methylation, acetylation and others) of biological materials (e.g., proteins or nucleic acids), one can carry out the necessary steps to determine if a site-specific or non-specific modification has occurred. Indeed, there exist or may be developed other assays that quantify modifications or provide an indication of the presence or absence of a material that may be used in accordance with embodiments of the present invention.

In an embodiment of the present invention, replicates of each sample are analyzed, e.g. two, three, or more replicates. Each xMap bead within the sample replicate is taken to be a single data point comprising doublet discrimination data, barcode classification data and reporter emission data. The dataset obtained from each sample replicate is subjected to a SAxCyB algorithm according to embodiments of the present invention in order to find differences of statistical significance between quantities of multiple analytes present in any given sample.

Each dataset is obtained from sampling a pool of xMap beads, typically but not exclusively derived from a single set of experimental conditions, e.g., a single well, and comprises reporter emission data points from a plurality of beads, e.g., from at least about 10, at least about 50, at least 100, or more beads for each barcode. As noted, there is at least one replicate for each trial, which is conveniently obtained by analyzing multiple wells for each test condition.

In an embodiment of the present invention, the analysis is multiplexed, that is, each sample is analyzed for at least 2 analytes of interest, at least 25 analytes of interest, at least 35 analytes of interest, at least 50 analytes of interest, or more. The data are then subjected to (a) an iterative minimization of error algorithm that takes into account variance between the datasets obtained from replicates, for example using least squares, least absolute error, etc.; and (b) a monotone transformation algorithm that stabilizes the variability of the data and considers background measurements. The resulting data are subjected to a bioequivalence-type test for determination of p values. The equivalence margin for the bioequivalence-type test is determined through a data-driven power estimation process.

In another embodiment of the present invention, however, single-plex analysis is implemented with no modifications.

The SAxCyB method as an embodiment of the present invention is based on the linear model where i indexes treatment; j indexes repeat for treatment i; and k indexes bead for treatment i within repeat j. With measured fluorescence intensity (FI); FI yᵢⱼₖ:

T(yᵢⱼₖ−βᵢⱼ)=μ+αᵢ+εᵢⱼₖ, k=1, . . . , nij, j=1, . . . , Ri, i=0, 1, . . . , N, (Eq. 1)

where N is the number of treatments; Rᵢ is the number of repeats for treatment i; and nᵢⱼ is the number of beads for treatment i within replicate j. μ is the overall mean. {αᵢ} are the effects of treatment i, which is the quantity of a main interest of the present disclosure. Inference on {αᵢ} pertains to the experimental question. {βᵢⱼ} represent the effects of repeats for treatment i.

T(•) is a monotone transform that stabilizes the variability of FI. {εᵢⱼₖ} are the errors in the transformed model that are independent and such that E(εᵢⱼₖ)=0 and Var(εᵢⱼₖ)=σᵢ². A common variance is assumed across the repeats j=1, . . . , Rᵢ for treatment i since they come from the same sample.

In some embodiments of the invention, the monotone transformation is the logarithmic transform

T(•)=log(•−M_{SB}+s) (Eq 2)

where M_{SB} is the pooled 5% trimmed mean blank measurements (SB) of the analyte, and s is a number that makes the internal term of the log positive for all k. It should be noted that blank measurements can implemented in the same tube as a sample. In certain implementation, these control beads are called “Reagent Blank Beads.” A device that uses such beads is the BioPlex 2200 form Bio-Rad.

The model (Eq. 1) suggests a standard weighted least squares method for estimating the parameters {αᵢ}: Given previously estimated {βᵢⱼ} and s, σᵢ² can be estimated empirically and weight T(yᵢⱼₖ−βᵢⱼ) proportionally to 1/σᵢ². For {αᵢ} to be defined uniquely, α₀=0 is set. {βᵢⱼ} is estimated using a nonlinear least squares method. It is required that Σⱼβᵢⱼ=0 in order to for {βᵢⱼ} to be defined uniquely.

The transformed data are tested for significance using a bioequivalence-type test comparing the treatment samples to the control samples. In testing multiple cases against one control, the null hypothesis is that the treatments are equivalent to the control:

Hi:|αᵢ−α₀|≦Δ, i=1, . . . ,N, (Eq. 3)

where Δ is the equivalence margin of the test. For each i, (Eq. 3) can be tested using two one-sided t-tests₃, resulting in a decision rule

Accept Hᵢ if T_{L,i}≧t_{α,v} and T_{U,i}≦t_{1-α,v}.

Reject Hᵢ otherwise, (Eq. 4)

This decision rule yields the p-value-like score

pᵢ=min(Fᵥ(T_{L,i}), 1−Fᵥ(T_{U,i})), (Eq. 5)

where Fᵥ(t) is the distribution function of a random variable following the t-distribution with v degrees of freedom is less than t. This score is designed such that the smaller of the pᵢ is, the more significant the instance i is.

In some embodiments, the data analysis is a computer-implemented method, where a computer is configured to perform the analytic steps. In some embodiments, the output signals generated from fluorescence emitted by the microparticles are to determine an identity of the microparticles, and information about the presence of the reporter on the surface of the microparticles. Therefore, the selection of the detectors and the spectral filters may vary depending on the type of dyes incorporated into or bound to the microparticles and/or the analyte/report being measured. The values generated by detection systems are used in the methods described herein.

A system for performing the analytic methods of the present invention is configured to analyze microparticle data according to embodiments described herein. In some embodiments, the system may include storage medium. Storage mediums may include program instructions. In some embodiments, a processor is configured to analyze microparticle data in combination with data acquired for the microparticle. In this manner, a processor of a measurement system may be configured to perform the data analysis described herein. Alternatively, a processor that is not a part of the measurement system but is coupled to the measurement system (e.g., by a transmission medium) such as a processor of a stand-alone computer system may be configured to analyze microparticle data as described herein.

Program instructions implementing methods such as those described herein may be transmitted over or stored on a storage medium. The storage medium may include, but is not limited to, a read-only memory, a random access memory, a magnetic or optical disk, or a magnetic tape. In an embodiment, a processor may be configured to execute the program instructions to perform a computer-implemented method according to the above embodiments. The processor may take various forms, including a personal computer system, mainframe computer system, workstation, network appliance, Internet appliance, personal digital assistant (PDA), a digital signal processor (DSP), field programmable gate array (FPGA), or other device. In general, the term “computer system” may be defined broadly to encompass any device having one or more processors that executes instructions from a memory medium. The program instructions may be implemented in any of various ways, including procedure-based techniques, component-based techniques, and/or object-oriented techniques, among others. For example, the program instructions may be implemented using ActiveX controls, C++ objects, JavaBeans, Microsoft Foundation Classes (“MFC”), or other technologies or methodologies, as desired.

It will be appreciated by those skilled in the art having the benefit of this disclosure that this invention is believed to provide computer-implemented methods, storage mediums, and systems for microparticle data analysis. Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following

This invention will be understood better by reference to the Examples that follow, but those skilled in the art will readily appreciate that the information detailed is only illustrative of the invention as described more fully in the claims that follow thereafter.

### EXPERIMENTAL

### Example 1

Luminex assays use large numbers of xMap beads to measure abundance of analytes. Conventional standard curve based analysis methods have limitations in detecting low or high abundance. Here SAxCyB (Significance Analysis of xMap Cytokine Beads) is presented as an embodiment of the present invention that implements a method that uses fluorescence measurements of individual beads to find significant differences between experimental conditions. SAxCyB as an embodiment of the present invention is shown to outperform conventional analysis schemes in both sensitivity (low fluorescence) and robustness (high variability).

In recent years, high throughput analysis of various analytes was made possible through xMap beads technology. An embodiment of the present invention, focuses on xMap cytokine assays, in which current methods allow for simultaneous (or multiplex) analysis of more than 50 different cytokines and other secreted proteins. The xMap bead offers the solid phase for a fluorescence ELISA (Enzyme-Linked Immunosorbent Assay). The analyte is classified through a two-color barcode embedded in the bead and the abundance of the analyte on the bead is determined by the fluorescent emission of the dye phycoerythrin on detection antibodies. Measured levels of fluorescence from the known cytokine dilutions are used to create standard curves, which then allow median fluorescence intensities (MFIs) to estimate unknown concentrations of analytes.

Currently, statistical analysis of xMap cytokine assays relies on repeat wells done in the assay and point estimators, usually the concentrations transformed from the MFIs, for each analyte within each well. This approach may be adequate when a large difference exists and where coefficients of variation are fairly small. But it is the nature of screening assays that many analytes have low fluorescence values and are, therefore, often reported as undetected. For example, this can happen when an MFI is computed to be below the intersection of the standard curve and the ordinate. These undetected values lead to sparse results, do not provide good estimation of the levels of analytes, and do not offer the best value for the cost of the assay.

Among other things, disclosed herein is a statistical approach for analysis of xMap cytokine data. Embodiments of the present invention can benefit from direct statistical analysis of fluorescence intensities (FIs). The use of individual bead fluorescence, as opposed to any summary number, provides access to low signal or poor quality data and allows more power to testing differences in analytes.

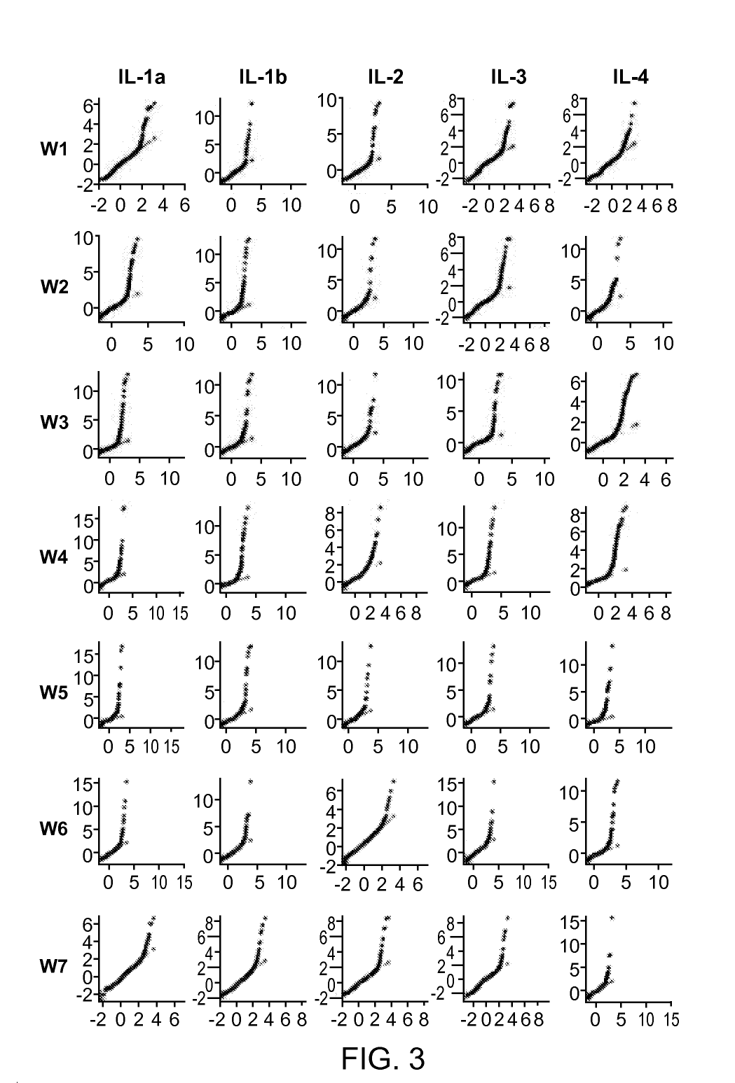

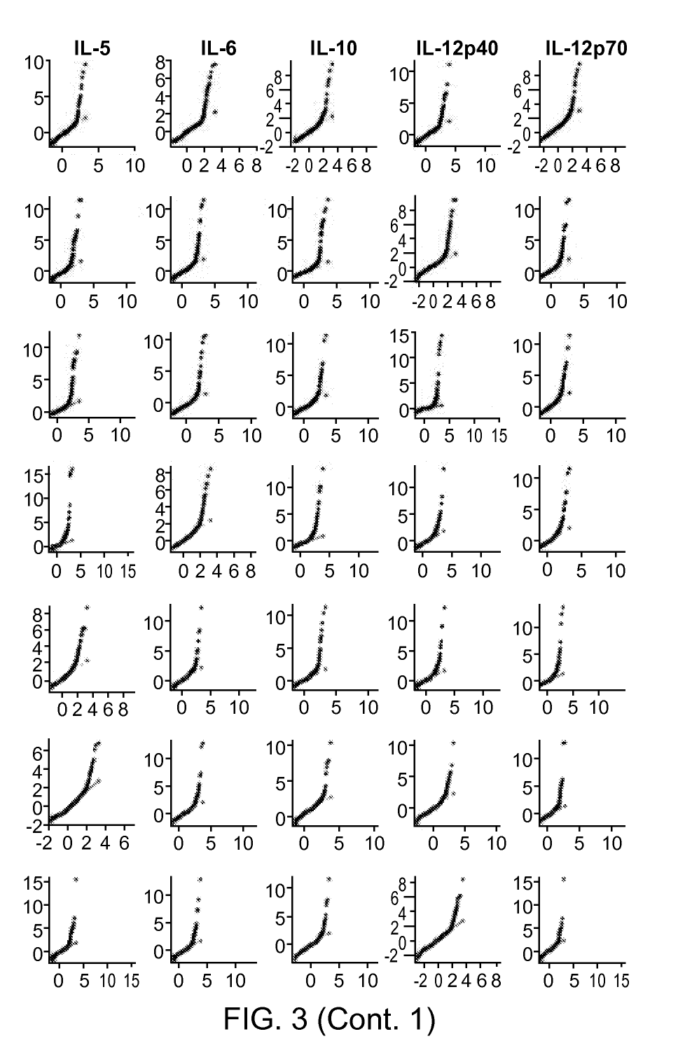

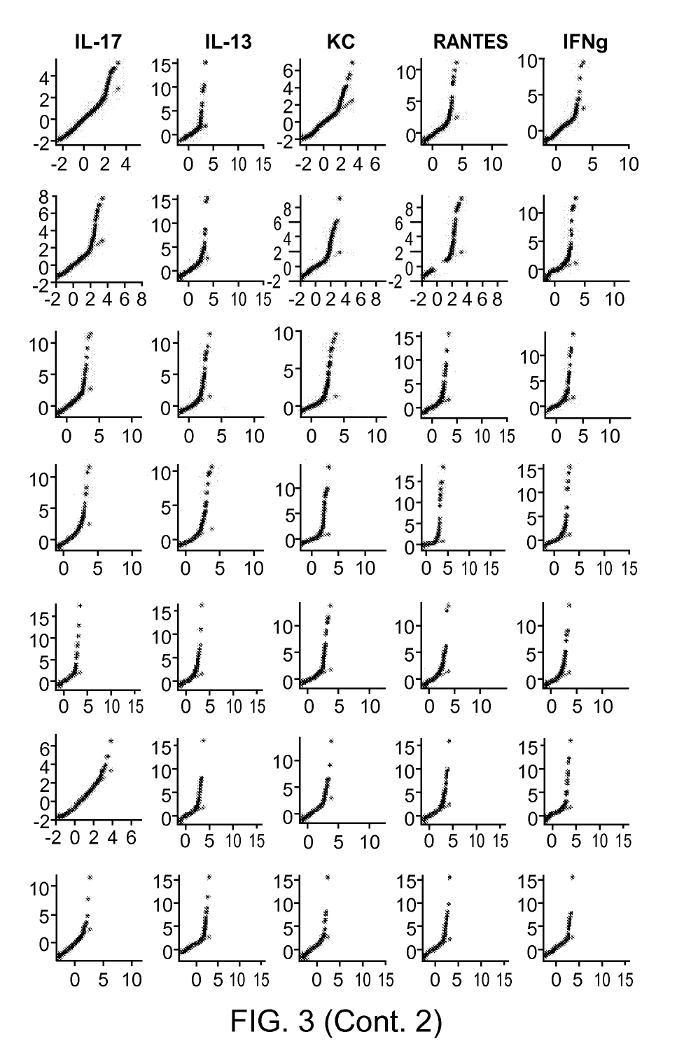

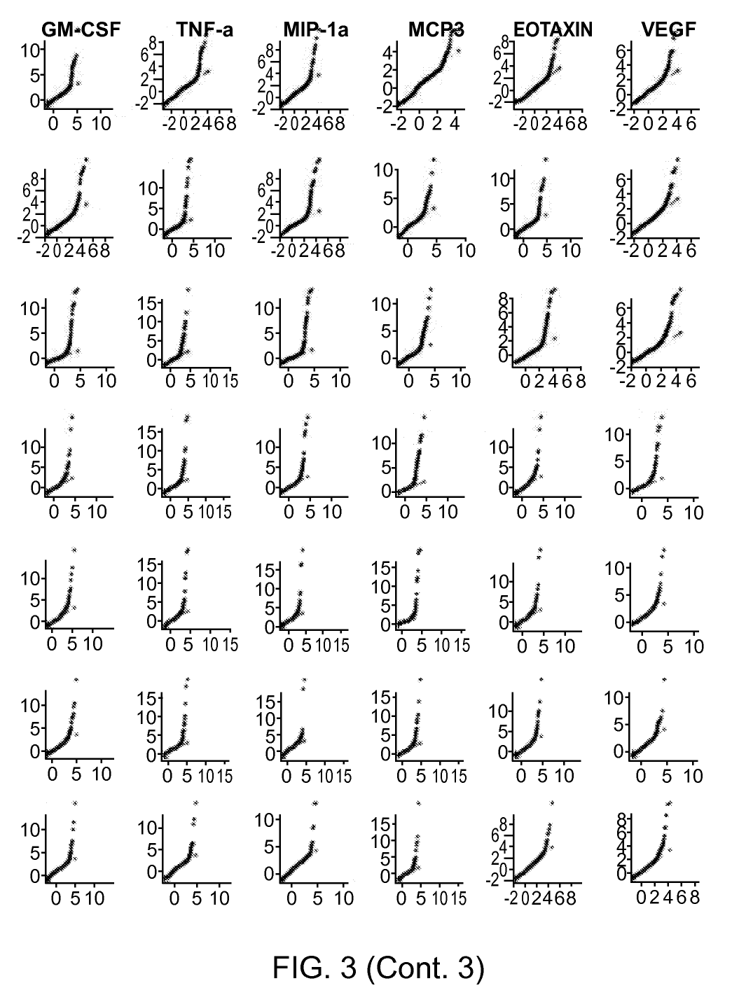

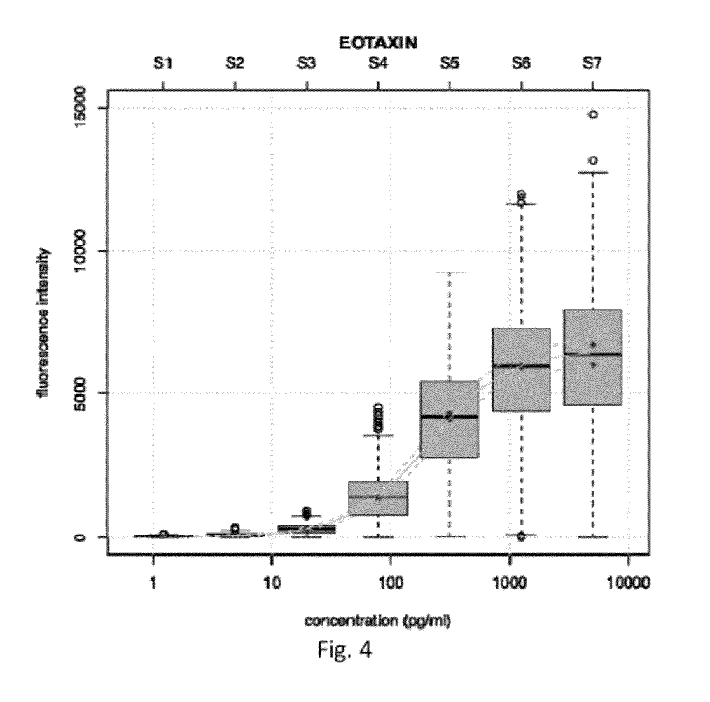

Upon examining individual bead fluorescence data of xMap cytokine assays (as obtained with Luminex machines, Luminex Inc., Austin, Tex., USA), it was observed that they are highly non-normal (see FIG. 3) and their variances are also quite variable across conditions (see FIG. 4).

For example, shown in FIG. 3 are quantile-quantile plots of typical xMap bead fluorescence intensity (FI) data for 21 standard analytes. The shown straight lines in the various graphs represent the standard normal distribution from which it is observed that the collected data varies.

Shown in FIG. 4 are exemplary scale of xMap bead fluorescence intensity data for eotaxin as observed in an application of the present invention. As shown, the standard concentration levels are 1.22, 4.88, 19.53, 78.12, 312.5, 1250, and 5000 pg/ml; ×4 diluted 7 times. The range of data is depicted as the box-and-whisker plot. Also shown are the MFIs in repeat wells and the confidence interval (dashed curves) of the standard curve (solid curve) fitted for the MFIs using a 4PL model (done with calib package for R, The R Project for Statistical Computing).

At lower fluorescence levels this is partly due to a background subtraction feature that allows multiplexed no-wash assays and, as a byproduct, introduces abnormally high variance. For these reasons, analyses based on t-statistics applied to MFIs are inappropriate for finding significant differences between samples. Statistical Analysis of xMap Cytokine Beads (SAxCyB) as an embodiment of the present invention incorporates the heavy-tailed distribution and the high variability of the data.

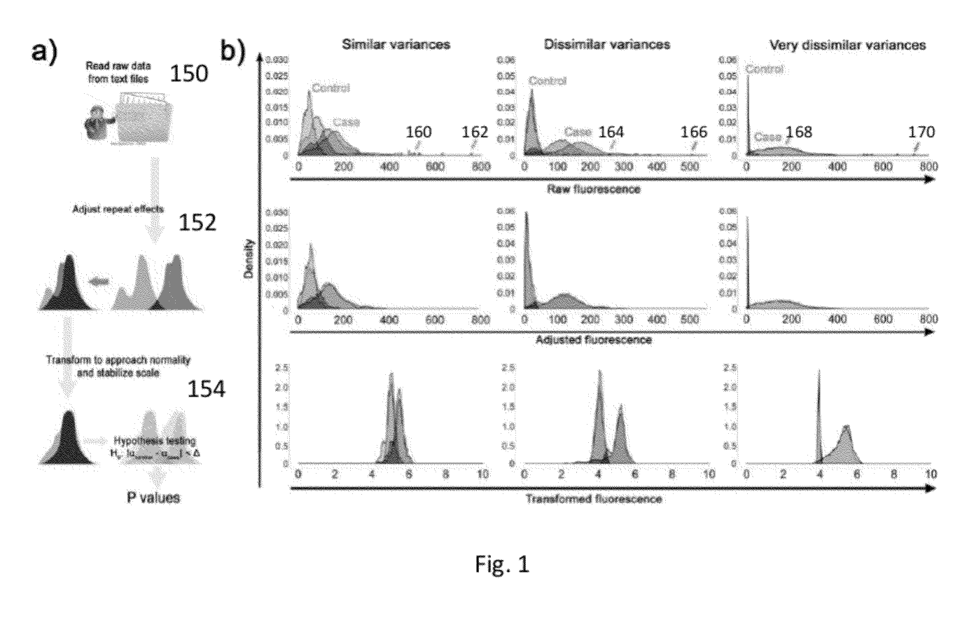

Shown in FIG. 1a is a schematic flow chart illustrating a method according to an embodiment of the present invention. For example, in an embodiment, a method of the present invention reads in raw Luminex data that may be in a text file at step 150. At step 152, in the model of the present invention, repeat wells of a common condition are combined after adjusting differences. At step 154, conditions of interest (henceforth cases) with large variances are compared simultaneously to the reference conditions (henceforth controls) after taking a variance-stabilizing transform.

Shown in FIG. 1b, are various applications of a method according to an embodiment of the present invention for experiments with different variances. Arrows 160 through 170 point to extreme outliers. As shown, FIG. 1b assists in visualizing the method's application to experimental data over different variances of case and control. The comparisons shown in FIG. 1b are made through a bioequivalence-type hypothesis testing, in which the equivalence margin is determined in a data-driven manner.

The performance of decision rules for comparing samples can be evaluated by measuring true positive rates (TPRs) and false positive rates (FPRs). To do this, an experiment was conducted in which assay standards were measured (used to generate the standard curve) since they are the most accurate sources of known amounts of cytokines for Luminex assays. Seven four-fold serial dilutions of assay standards were performed in seven repeats each. The resulting cytokine concentrations (or instances) range from saturation (5000 pg/ml) to the lower detection limit (1.22 pg/ml). Blank wells with sample buffer only (SB) were also included.

For the present analysis, a set of in-silico experiments were created. Each in-silico experiment consists of two components. First, to test FPR, a pair of repeats were randomly designated from one instance as control and two random pairs as cases. Since both control and cases came from the same instance, it is expected not to reject the null hypothesis (that they are not significantly different). Second, to test TPR, three random pairs of repeats from another instance were designated as cases. This was done for all six instances other than the control. Since the control and cases came from different instances, it is expected to reject the null hypothesis.

In the present example, 315 analyses were generated for each instance, yielding 630 true negatives and six groups of 945 true positives. SAxCyB according to an embodiment of the present invention was applied to test significant differences at nominal significance levels of 0.01 and 0.05, and counted false positives and true positives to compute FPR and TPR respectively. This analysis was conducted for each of the 51 analytes (Supplementary Table 1). As reference decision rules, a two-sample t-test (“t_MFI”) was used that employs only MFIs (therefore two measurements for each instance) and a two-sample t-test (“t_fullFI”) that employs all bead measurements and pools repeats. The first reference is a common analysis method of xMap data and the second is a naïve use of all the individual bead measurements.

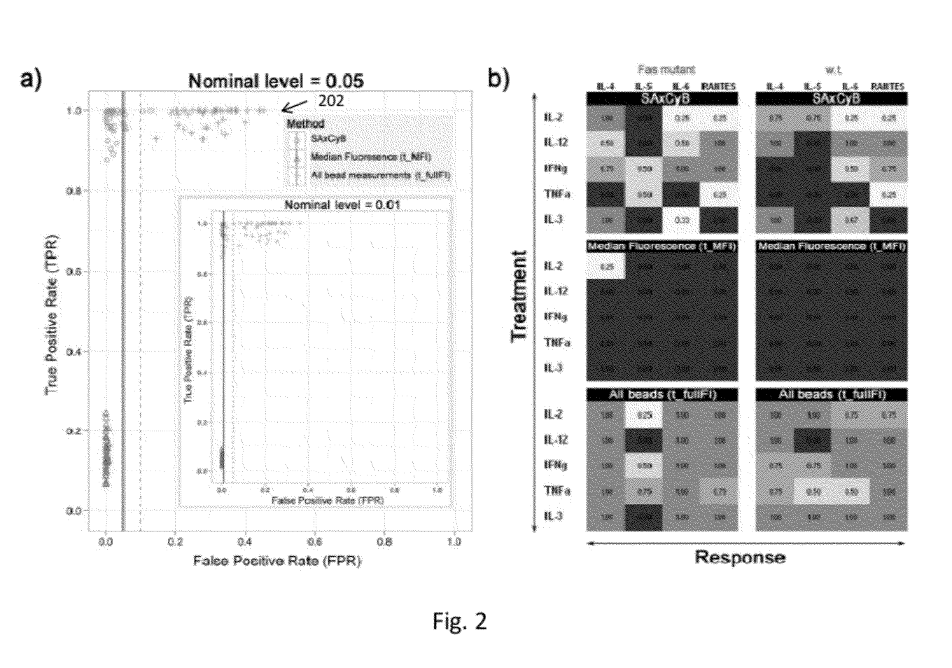

Shown in FIG. 2 are certain graphs that demonstrate the performance of SAxCyB as an embodiment of the present invention. More particularly, shown in FIG. 2a is a graph of False Positive Rate (FPR) versus True Positive Rate (TPR) to assist in an evaluation of SAxCyB as an embodiment of the present invention using diluted standard analytes. False Positive Rates and True Positive Rates of were computed per analyte for SAxCyB as an embodiment of the present invention and reference methods t_fullFI and t_MFI. All numbers were adjusted for multiple comparisons. As shown the solid vertical line indicates a nominal significance level; 0.05 (outer panel), 0.01 (inner panel). The dashed vertical line indicates 0.10 (outer panel) and 0.05 (inner panel).

Pooled across the full scale of data, SAxCyB, according to an embodiment of the present invention, achieved higher TPR (True Positive Rate) with lower FPR (False Positive Rate) as shown by arrow 202 for the data points clustered on the upper-left corner in the graph of FIG. 2a than the other two reference decision rules. At the nominal level of 0.05, 50 out of 51 analytes had FPRs less than the nominal level; the remaining analyte had FPRs less than 0.10. At the 0.01 level, 48 analytes had FPRs less than the nominal level; the other three had FPRs less than 0.05. TPRs were very high for both levels: all of them were greater than 0.85, most of them were very close to 1. Naively using individual bead fluorescence (t_fullFI) also yielded high TPRs, but had much higher FPRs. At the level 0.05, all analytes had FPRs greater than 0.10; at 0.01, all FPRs were greater than 0.05. This can be a consequence of the heavy tails and/or the discrepant scales of FIs that usually degrades the performance of a two-sample t-test. SAxCyB, in an embodiment of the present invention, overcomes this by adjusting the repeat effects and by variance-stabilizing transformation. As can be expected by the small number of data points, t_MFI had unacceptably low TPRs.

Shown in FIG. 2b are certain graphs that illustrate the use of SAxCyB as an embodiment of the present invention in analysis of a cytokine stimulation assay. As shown, T cells from Fas mutant or wild type mice were treated with the indicated cytokines, and cytokine synthesis in these cells was measured (IL-4, IL-5, IL-6 and RANTES are shown). Treatments were done in 4 (IL-2, IL-12, IFNγ and TNFα) or 3 (IL-3) doses. A fraction of the responding doses is shown.

In order to investigate how SAxCyB, according to an embodiment of the present invention, performs for real experiments, it was used to analyze mouse cytokine production in response to different treatments. Treated cells were compared to untreated cells. Data were generated from 80 wells for samples and 16 wells for the standard curve. The 80 wells were divided into two sets of 40, where each set consists of one control (untreated) and 19 treatments in 4 or 3 doses, all technically repeated in duplicates. Twenty one analytes were measured per well. Each well contained 5,791±601 data points, each bead/well combination contained 276±79 events.

Table 1 shows that, in an embodiment of the present invention, SAxCyB calls 238 differences (at the 0.05 significance level) compared to 38 and 44 found by commercial software (BeadView and MasterPlex QT respectively). Many of the calls were for data at low MFIs. Note that more than a half of the analyzed data points were thresholded out in the commercial package, whereas SAxCyB, according to an embodiment of the present invention, did not suffer such difficulties by construction.

TABLE 1
Instance-by-instance sensitivity analysis results.

| | | MasterPlex | |
|---|---|---|---|
| | BeadView | QT V4 | SAxCyB |
| Individual data points | 1680 | 1680 | 555,981 |
| analyzed | | | |
| Data points thresholded out | 1224 | 1002 | NA |
| (due to standard curve | | | |
| fitting) | | | |
| Significant differences | 38* | 44* | 238† |

*t-test, α = 0.05 (no multiple hypothesis correction).
NA-Not applicable.
†two one-sided t-tests, α = 0.05 (step-up multiple hypothesis correction).

In a more specific analysis of a subset of this data, Fas mutant and wild type mice T helper cells were treated with IL-2, IL-12, IFNγ, TNFα and IL-3 at various doses or untreated. Shown are 4 response cytokines (IL-4, IL-5, IL-6 and RANTES). SAxCyB according to an embodiment of the present invention (top), t_MFI (center) and t_fullFI were used to call differences between treated and untreated. While t_MFI displayed low sensitivity, calling only one difference, t_fullFI was overly sensitive, calling most (125 of 152) comparisons different. Hence both methods resulted in effectively uninterpretable data. In an embodiment of the present invention, SAxCyB captured small differences (65 of 152) while maintaining overall interpretability of the data and revealing differences between the mutant and wild type mice. This result demonstrates the importance of deriving the sensitivity threshold (Δ, see Methods) from the experimental data. SAxCyB, as an embodiment of the present invention, outperforms the other two methods by far in the present context.

SAxCyB as an embodiment of the present invention is a statistical model that relies on the individual fluorescence measurements of xMap beads and repeat effects to estimate significant differences between experimental conditions.

Although the approach was constructed using Luminex cytokine assays, it can be easily extended to all xMap assays since the common denominators of conjugated beads and fluorescence readouts behave similarly.

Methods

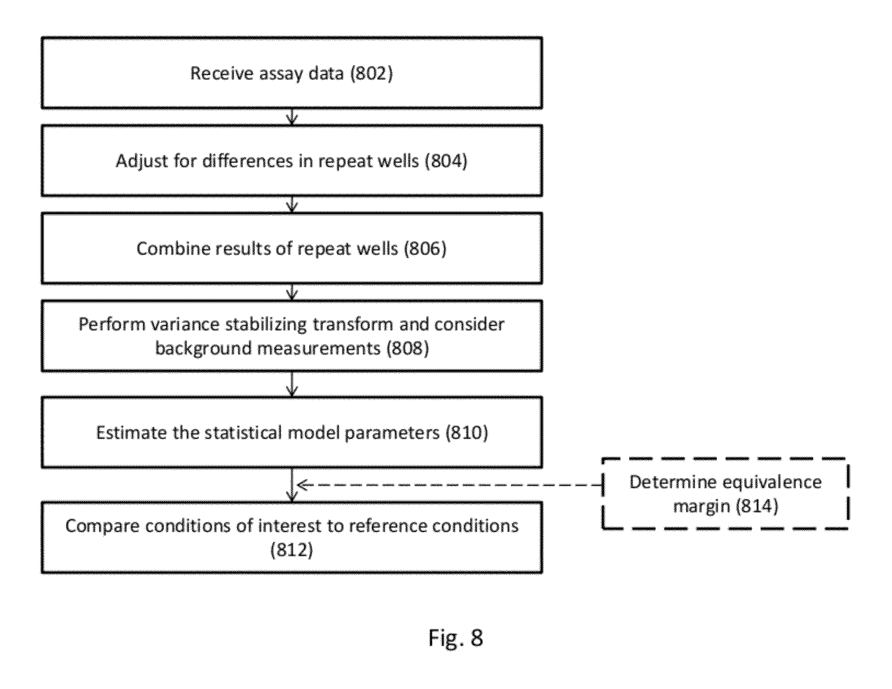

Shown in FIG. 8 is a method according to an embodiment of the present invention. As shown, at step 802, assay data is received that will subsequently be analyzed. In an embodiment of the present invention, data from a Luminex apparatus is received, however, it is to be understood that other types of data may be used as known to one of ordinary skill in the art. For example, other data derived from fluorescence-based bead testing may be used. More broadly, data from other bead-based systems may be used. Indeed, the teachings of the present invention can be implemented in testing systems and methods as they continue to be developed.

A characteristic of an application of the present invention is that repeat wells are implemented. So as to account for this effect, in an embodiment of the present invention as shown at step 804, an adjustment is applied for differences in repeat wells. Then, at step 806, the results of the various repeat wells are combined.

Importantly, in an embodiment of the present invention, the received data is not characterized as behaving according to a standard normal distribution. Indeed, the data may appear to vary widely with seemingly no consistent patterns. Advantageously, however, after the application of a predetermined transform, the transformed data was observed to have a stabilized variance. Accordingly, at step 808, a predetermined variance stabilizing transform is applied. In the disclosure further below, a particular logarithm-based variance stabilizing transform will be discussed, however, one of ordinary skill in the art will understand that other types of transforms may be applicable for different types of data. For example, in certain other embodiments a inverse (e.g., 1/x) transform may be applicable. In still another embodiment, a transform of the form xⁿ (where n is not equal to 1) may be applied. Another embodiment can use Box-Cox transform family or Huber transform (see Huber P J (1981) Robust statistics (John Wiley and Sons). Many other transforms can be used so long as the transform tends to stabilize the observed variance of the data being processed.

In another embodiment of the invention, background measurements can also be considered along with the variance stabilizing transform so as to provide improved results. For example, background or “blank” measurements can be used to determine machine, measurement, and other errors.

With the transformed data, at step 810, the parameters for the statistical model are estimated. For example, in an embodiment of the invention, mean and standard deviation are computed. Other statistical parameters can also be computed as would be obvious to one of ordinary skill in the art.

In an alternative embodiment, a further step 814 is performed in which a power estimation procedure is used to determine the equivalence margin for a bioequivalence test by comparing conditions of interest to certain reference conditions.

At step 812, a method according to an embodiment of the present invention compares conditions of interest to certain reference conditions so as to evaluate the data being processed. In a particular embodiment that implements step 814, step 812 may also include applying a bioequivalence-type hypothesis testing with the equivalence margin determined in 809. In other embodiments of the present invention, other types of hypothesis testing can be applied as known to those of ordinary skill in the art.

Further details of a particular embodiment will now be described that will assist in the understanding of the broader and general concepts of the present invention. Those of ordinary skill in the art will understand, however, that the disclosed embodiments are exemplary and are not limiting. Indeed, one of ordinary skill in the art will be aware of changes and modifications that remain within the teachings of the present invention.

Mice: In an application of an embodiment of the present invention, testing was performed on mice. All mice were obtained from Jackson laboratories and were maintained in the Stanford research animal facility according to ACUC guidelines.

Luminex assays: In an embodiment of the present invention, data was collected using Luminex assays. For the mice cytokine experiment, CD4 T cells from two strains of mice, Fas mutant (MRL.MpJ.lpr) and wild type (MRL.MpJ) were stimulated for 16 hours with subsequent blocking of golgi-mediated secretion. The cells were lysed and intracellular cytokines were measured by Luminex using mouse 21 plex beads. All Luminex experiments were performed by the Stanford Human Immune Monitory Center using Panomics beads and Luminex 100 IS or Luminex 200 machines.

The SAxCyB model according to an embodiment of the present invention: The SAxCyB method, according to an embodiment of the present invention, as used here is based on the following linear model. Let i indexes treatment; j indexes repeat for treatment i; and k indexes bead for treatment i within repeat j. With measured FI yᵢⱼₖ, we write

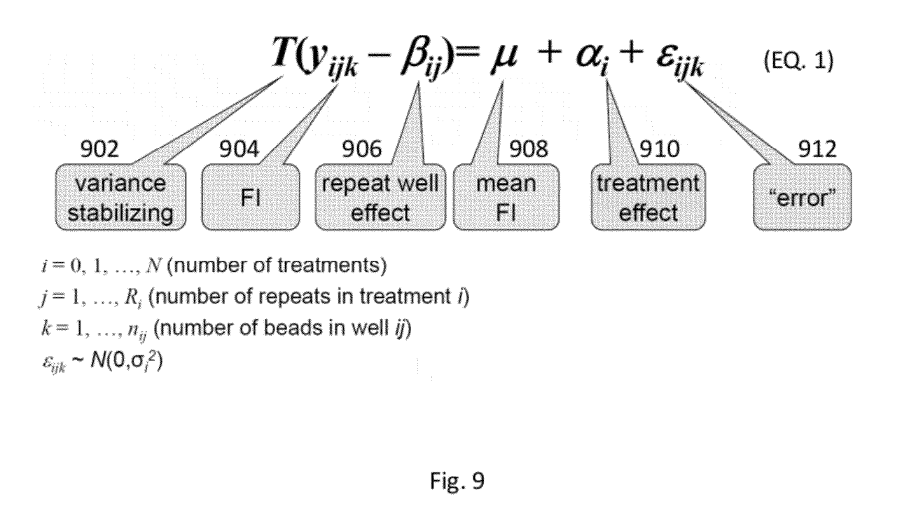

T(yᵢⱼₖ−βᵢⱼ)=μ+αᵢ+εᵢⱼₖ, k=1, . . . ,nᵢⱼ, j=1, . . . ,Ri, i=0,1, . . . ,N, (Eq. 1)

where N is the number of treatments; Rᵢ is the number of repeats for treatment i; and nᵢⱼ is the number of beads for treatment i within replicate j. Shown in FIG. 9 is a graphical representation of the various components of Eq. 1. μ (908 as shown in FIG. 9) is the overall mean. {αᵢ} (910) are the effects of treatment i, which is the quantity of a main interest. Inference on {αᵢ} pertains to the experimental question. {βᵢⱼ} (906) represent the effects of repeats for treatment i (e.g., repeat wells). T(•) (902) is a monotone transform that stabilizes the variability of FI. {εᵢⱼₖ} (912) are the errors in the transformed model that are independent and such that E(εᵢⱼₖ)=0 and Var(εᵢⱼₖ)=σᵢ² (e.g., normal distribution). Equal variance is not assumed here since even after the transform the variance may still vary as i varies. However, a common variance is assumed across the repeats j=1, . . . , Rᵢ for treatment i since they come from the same sample. Simply put, it is assumed the FI (904) measurements adjusted for the repeat effect follow an ANOVA model.

A logarithmic transform was empirically found that yields good performance across the scale of possible measurements:

T(•)=log(•−M_{SB}+S) (Eq. 2)

Here, M_{SB} is the pooled 5% trimmed mean blank measurements (SB) of the given cytokine, and s is a number that makes the internal term of the log positive for all k. M_{SB} roughly determines the precision of the measurement. Since blank measurements are standard in every experiment, it is convenient to use it to adjust the FI. Note that a similar transform was used for probe-level analysis of expression microarrays.

Further note that other transforms (e.g., 1/x, xⁿ, etc.) may be applicable in other types of data as known to those of ordinary skill in the art.

Estimation: In an embodiment, a standard weighted least squares method for estimating the parameters {αᵢ} is used: Given previously estimated {βᵢⱼ} and s, σᵢ² is estimated empirically and weight T(yᵢⱼₖ−βᵢⱼ) proportionally to 1/σᵢ². For {αᵢ} to be defined uniquely, α₀=0 is set. (Only differences are of interest.) {βᵢⱼ} can be estimated using a nonlinear least squares method. This requires a good initialization, for which satisfactory results were found with Huber robust regression on repeats for each treatment. Often this initialization is good enough. Σⱼβᵢⱼ=0 is required in order to for {βᵢⱼ} to be defined uniquely. This condition also imposes symmetry that is convenient for testing treatment effects.

Hypothesis testing using SAxCyB as an embodiment of the present invention: The treatment samples were compared to the control samples using a bioequivalence-type test. In testing multiple cases against one control, the null hypothesis is that the treatments are equivalent to the control:

Hi:|αᵢ−α₀|≦Δ, i=1, . . . ,N, (Eq. 3)

where Δ is the equivalence margin of the test. For example, setting Δ=0.05 in (Eq. 3) tests whether the treatment is within 5% margin of the control. If not all of the treatments are equivalent to the control, it is desirable to know which treatments significantly differ from the control. For each i, (Eq. 3) can be tested using two one-sided t-tests₃, resulting in a decision rule

Accept Hᵢ if T_{L,i}≧t_{α,v} and T_{U,i}≦t_{1-α,v}.

Reject Hᵢ otherwise, (Eq. 4)

where T_{L,i}=(αᵢ−α₀+Δ)/s and T_{U,i}=(αᵢ−α₀−Δ)/s; αᵢ and α₀ are the estimated effects of the case and the control, and s is the estimated normal theory standard deviation of their difference having v degrees of freedom (Δ can be thought of as the tolerance to the deviation from the normality of the data). These values are obtained from fitting the linear model (Eq. 1) to data.

This decision rule yields the p-value

pᵢ=min(Fᵥ(T_{L,i}), 1−Fᵥ(T_{U,i})), (Eq. 5)

where Fᵥ(t) is the probability that a random variable following the t-distribution with v degrees of freedom is less than t.

Note that when testing all the hypotheses simultaneously, the rate of false positives (type I error) inflates. The two one-sided t-tests procedures were used followed by a post-hoc adjustment for controlling family-wise error rate. The false discovery rate control₉ was used at the user's discretion for cases in which the number of comparisons is large(e.g., N greater than 20). When there are multiple controls each of which has multiple cases, testing (Eq. 3) was repeated independently for each case-control group.

Selecting Δ: In choosing Δ, t is desirable to let the data choose Δ in an embodiment of the present invention. In an embodiment, data-driven machinery such as SAM is used. In such an embodiment, there is not the luxury of possessing a few hundred of p-values as microarray analyses for which SAM is designed. Instead, Δ was chosen at which the estimated power is reasonably high. The power of the decision rule (Eq. 4) is given as

π_{I}(αᵢ−α₀,σ,v;Δ)=1−Pr{T_{L,i}≧t_{αv} and T_{U,I}≦t_{1-α,v}|αᵢ−α₀,σ,v}

Under the assumption that αᵢ−α₀ follows a normal distribution N(αᵢ−α₀, σ), the vector (T_{L,i}, T_{U,I}) has a bivariate noncentral t-distribution with v degrees of freedom and noncentrality parameters

δ_{L,i}(Δ)=(α_{I}−α₀+Δ)/σ and δ_{U,i}(Δ)=(α_{I}−α₀−Δ)/σ.

The power is estimated at the estimated effect size, i.e., evaluate π_{I} (aᵢ−a₀, s, v; Δ). This estimated power is a nonincreasing function of Δ. For each case-control group, the largest Δ (call this Δ*) is chosen such that the average estimated power (over i=1, . . . , N) is greater than a threshold. Then, the threshold varied and the median of the As that are selected are plotted, and find the inflection point. The threshold that yields the inflection point is used, and this threshold in turn determines Δ* for each case control group.

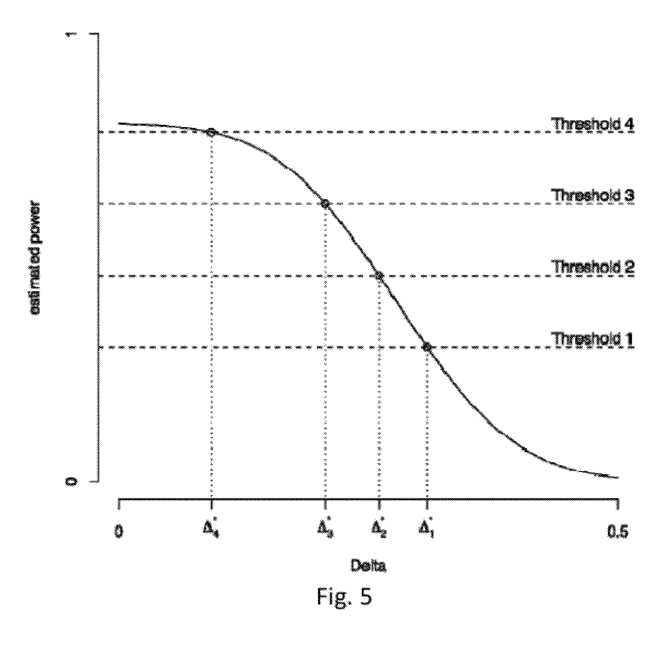

Shown in FIG. 5 is a graph that illustrates the tradeoffs associated with choosing an appropriate Δ using the power estimated from the data. For example, in an application of an embodiment of the present invention, increasing the threshold from Threshold 3 to Threshold 4 yields the largest change in Δ. Other applications, however, may yield different results.

Excluding outliers: Extreme outlier measurements can occur due to contributions of carryover beads from previous wells. In an embodiment, outliers were excluded in the measured FIs from the analysis. FI measurements greater than 5% trimmed mean plus 4 times standard deviation (also 5% trimmed) were considered outliers. This is done for all methods compared in this text.

Computational resources: Statistical analyses were performed with the R statistics package and MATLAB 2007a/2009 ran on 8-core x86-64 GNU/Linux server and on a Windows XP workstation, respectively.

List of analytes used for the sensitivity analysis: Analytes are sorted by the FPR of SAxCyB, according to an embodiment of the present invention. All numbers are MCP-adjusted at the nominal significance level 0.05. In the shaded area are the analytes whose SAxCyB FPR is greater than 0.05.

Also shown are a two-sample t-test (“t_MFI”) that employs only MFIs (Median Fluorescence Intensities; therefore two measurements for each instance) and a two-sample t-test (“t_fullFI”) that employs all bead measurements and pools repeats. The first reference is a common analysis method of xMap data and the second is a naïve use of all the individual bead measurements.

TABLE 2

| | SAxCyB | | t_MFI | | t_fullFI | |
|---|---|---|---|---|---|---|
| Analyte | FPR | TPR | FPR | TPR | FPR | TPR |
| GM-CSF | 0.0000 | 0.9789 | 0.0059 | 0.1354 | 0.3193 | 1.0000 |
| ICAM-1 | 0.0000 | 0.8597 | 0.0068 | 0.1258 | 0.1433 | 0.9283 |
| IFNa | 0.0000 | 0.9727 | 0.0027 | 0.1288 | 0.2036 | 1.0000 |
| IFNb | 0.0000 | 0.9513 | 0.0005 | 0.2031 | 0.2063 | 0.9895 |
| IL-17 | 0.0000 | 0.9800 | 0.0000 | 0.1225 | 0.3088 | 1.0000 |
| IL-17F | 0.0000 | 0.9602 | 0.0000 | 0.1816 | 0.1878 | 0.9927 |
| IL-12p40 | 0.0000 | 0.9765 | 0.0000 | 0.1841 | 0.2862 | 1.0000 |
| IL-12p70 | 0.0000 | 0.9912 | 0.0018 | 0.1220 | 0.3147 | 1.0000 |
| IL-10 | 0.0000 | 0.9903 | 0.0050 | 0.1548 | 0.3574 | 1.0000 |
| IL-8 | 0.0000 | 0.9881 | 0.0000 | 0.2432 | 0.2209 | 1.0000 |
| IL-7 | 0.0000 | 0.9708 | 0.0000 | 0.1091 | 0.4395 | 1.0000 |
| IL-6 | 0.0000 | 0.9769 | 0.0000 | 0.1036 | 0.3696 | 1.0000 |
| IL-5 | 0.0000 | 0.9731 | 0.0000 | 0.1575 | 0.3279 | 1.0000 |
| IL-4 | 0.0000 | 0.9822 | 0.0009 | 0.1463 | 0.4068 | 1.0000 |
| IL-1a | 0.0000 | 0.9752 | 0.0000 | 0.1241 | 0.4526 | 1.0000 |
| IL-1b | 0.0000 | 0.9816 | 0.0009 | 0.2330 | 0.3610 | 1.0000 |
| LIF | 0.0000 | 0.9846 | 0.0087 | 0.1663 | 0.2685 | 1.0000 |
| MCP-1 | 0.0000 | 0.9414 | 0.0009 | 0.1194 | 0.2721 | 1.0000 |
| MIG | 0.0000 | 0.9708 | 0.0045 | 0.1573 | 0.3188 | 1.0000 |
| MIP-1a | 0.0000 | 0.9394 | 0.0000 | 0.1973 | 0.1937 | 1.0000 |
| PAI-1 | 0.0000 | 0.9782 | 0.0032 | 0.1335 | 0.3728 | 1.0000 |
| RANTES | 0.0000 | 0.9590 | 0.0000 | 0.0924 | 0.3125 | 1.0000 |
| SCF | 0.0000 | 0.9721 | 0.0005 | 0.1333 | 0.3143 | 1.0000 |
| sFas-Ligand | 0.0000 | 0.9782 | 0.0000 | 0.0857 | 0.3755 | 1.0000 |
| TGFa | 0.0000 | 0.9414 | 0.0032 | 0.1636 | 0.3175 | 1.0000 |
| TGF-b | 0.0000 | 0.9353 | 0.0009 | 0.2168 | 0.3719 | 1.0000 |
| TNF-a | 0.0000 | 0.9897 | 0.0000 | 0.1239 | 0.3125 | 1.0000 |
| TNF-beta | 0.0000 | 0.9915 | 0.0027 | 0.1791 | 0.2485 | 1.0000 |
| TRAIL | 0.0000 | 0.9614 | 0.0005 | 0.2054 | 0.3052 | 0.9943 |
| VCAM-1 | 0.0000 | 0.9433 | 0.0000 | 0.0641 | 0.3370 | 0.9773 |
| VEGF | 0.0000 | 0.9702 | 0.0000 | 0.0933 | 0.2277 | 1.0000 |
| Eotaxin | 0.0002 | 0.9435 | 0.0028 | 0.1176 | 0.2236 | 0.9527 |
| HGF | 0.0005 | 0.9630 | 0.0027 | 0.0970 | 0.3370 | 0.9989 |
| CD40Ligand | 0.0007 | 0.9384 | 0.0018 | 0.1189 | 0.3342 | 0.9711 |
| M-CSF | 0.0007 | 0.9699 | 0.0000 | 0.1831 | 0.3565 | 1.0000 |
| FGF-Basic | 0.0011 | 0.9371 | 0.0014 | 0.0995 | 0.2884 | 0.9551 |
| MIP-1b | 0.0011 | 0.9569 | 0.0082 | 0.2043 | 0.2812 | 0.9986 |
| IP-10 | 0.0014 | 0.9576 | 0.0009 | 0.1530 | 0.3288 | 1.0000 |
| GRO-alpha | 0.0020 | 0.9034 | 0.0014 | 0.1742 | 0.2358 | 0.9890 |
| Resistin | 0.0023 | 0.9217 | 0.0000 | 0.0683 | 0.4005 | 0.9700 |
| ENA-78 | 0.0029 | 0.8854 | 0.0014 | 0.1113 | 0.2458 | 0.9426 |
| IL-13 | 0.0041 | 0.9774 | 0.0018 | 0.1487 | 0.2653 | 1.0000 |
| IL-15 | 0.0057 | 0.9368 | 0.0050 | 0.1186 | 0.2073 | 0.9624 |
| Leptin | 0.0066 | 0.8722 | 0.0023 | 0.1192 | 0.2780 | 0.9277 |
| G-CSF | 0.0084 | 0.9402 | 0.0009 | 0.1637 | 0.2875 | 0.9583 |
| IL-1Ra | 0.0091 | 0.8913 | 0.0005 | 0.0716 | 0.3433 | 0.9393 |
| IFNg | 0.0111 | 0.9588 | 0.0000 | 0.1365 | 0.2785 | 0.9927 |
| MCP3 | 0.0218 | 0.9468 | 0.0000 | 0.0847 | 0.3220 | 0.9682 |
| IL-2 | 0.0240 | 0.9784 | 0.0018 | 0.0654 | 0.4408 | 1.0000 |
| NGF | 0.0247 | 0.9162 | 0.0005 | 0.1222 | 0.3324 | 0.9477 |
| PDGF-BB | 0.0683 | 0.9879 | 0.0077 | 0.1223 | 0.3501 | 1.0000 |

Issues with MFIs: In principle, the median or the mean of a set of random numbers can be as efficient as using all measurements as the mean is a sufficient statistic for the location parameter under the normal assumption; and the median is known to be a robust estimate of location. The deviation from normality in the bead data was discussed. The sample mean is observed as very sensitive to the outliers or the scale of the bead distribution. The sample median has a problem with estimating its variance, as it is inversely proportional to the square of the probability density evaluated at the population median. Estimating the value of a density, let alone the reciprocal of its square, is notoriously difficult. No matter how one estimates the variance of the median, even for large sample sizes, a t-like statistic for comparing medians may be a poor choice by which to make comparisons, not least here, where sampling distributions are far from normal; and there are additional problems of scaling.

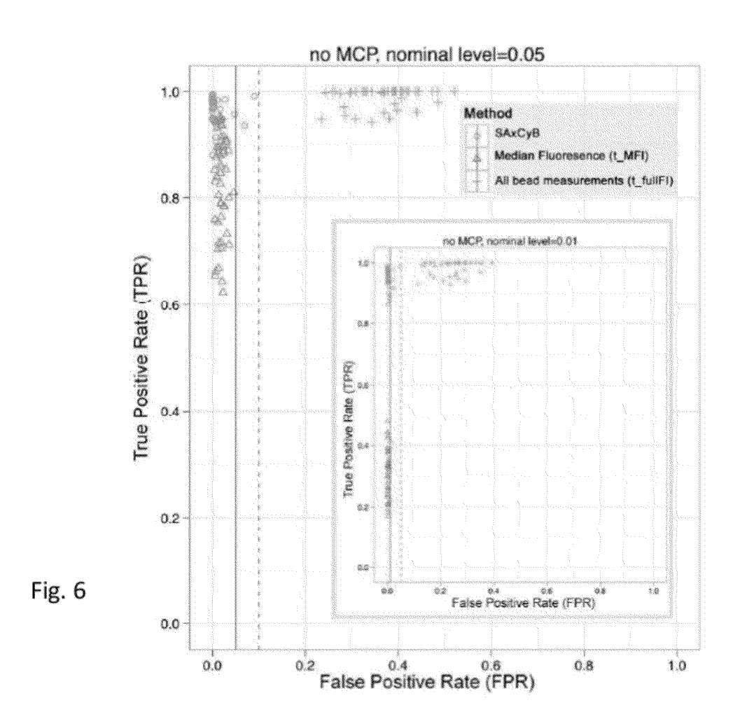

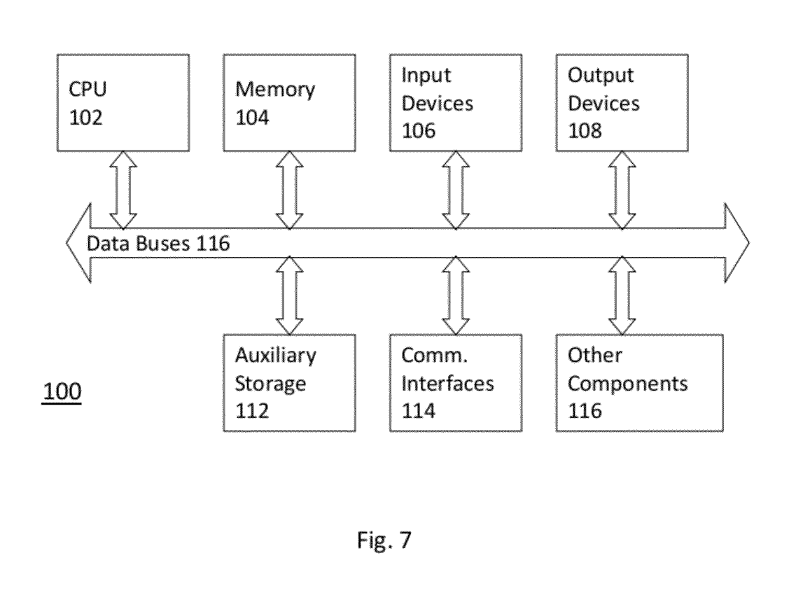

Adjustment for multiple comparisons: Multiple comparison procedure (MCP) is preferably used when comparing a control to many cases. Use of MCP not only reduces inflation in the type I error (FPR) but can make a decision rule for significant differences more or less immune to the nominal significance level. For example, in the sensitivity analysis from the previous section, it was observed that as the nominal level is decreased from 0.05 to 0.01, t_MFI, unadjusted for MCP, showed a dramatic reduction in TPR (see FIG. 6). As shown in FIG. 6, FPR versus TPR is charted for SAxCyB as an embodiment of the present invention, in comparison with t_fullFI and t_MFI at the nominal significance level 0.05 and 0.01 (inset). All numbers are not adjusted for multiple comparisons (MCP). This indicates that t_MFI yields many marginal unadjusted p-values around 0.05), severely affected by the change of nominal level or multiple comparison adjustment.

It should be appreciated by those skilled in the art that the specific embodiments disclosed above may be readily utilized as a basis for modifying or designing processing algorithms or systems. It should also be appreciated by those skilled in the art that such modifications do not depart from the scope of the invention as set forth in the appended claims.

The following references are herein incorporated by reference for all purposes.
1. Kellar, K. L. et al. Multiplexed fluorescent bead-based immunoassays for quantitation of human cytokines in serum and culture supernatants. Cytometry 45, 27-36 (2001).
2. Jacobson, J. W., Oliver, K. G., Weiss, C. & Kettman, J. Analysis of individual data from bead-based assays (“bead arrays”). Cytometry Part A 69A, 384-390 (2006).
3. Schuirmann, D. J. A comparison of the Two One-Sided Tests Procedure and the Power Approach for assessing the equivalence of average bioavailability. Journal of Pharmacokinetics and Pharmacodynamics 15, 657-680 (1987).
4. Fieller, E. C. Some Problems in Interval Estimation. Journal of the Royal Statistical Society. Series B (Methodological) 16, 175-185 (1954).
5. Creasy, M. A. Limits for the Ratio of Means. Journal of the Royal Statistical Society. Series B (Methodological) 16, 186-194 (1954).
6. Bolstad, B., Irizarry, R., Astrand, M. & Speed, T. A comparison of normalization methods for high density oligonucleotide array data based on variance and bias. Bioinformatics 19, 185-193 (2003).
7. Huber, P. J. Robust statistics. (John Wiley and Sons: 1981).
8. Holm, S. A Simple Sequentially Rejective Multiple Test Procedure. Scandinavian Journal of Statistics 6, 65-70 (1979).
9. Benjamini, Y. & Hochberg, Y. Controlling the False Discovery Rate: A Practical and Powerful Approach to Multiple Testing. Journal of the Royal Statistical Society. Series B (Methodological) 57, 289-300 (1995).
10. Tusher, V. G., Tibshirani, R. & Chu, G. Significance analysis of microarrays applied to the ionizing radiation response. Proceedings of the National Academy of Sciences of the United States of America 98, 5116-5121 (2001).
11. Phillips, K. F. Power of the two one-sided tests procedure in bioequivalence. Journal of Pharmacokinetics and Pharmacodynamics 18, 137-144 (1990).
12. Hanley, B. Variance in multiplex suspension array assays: carryover of microspheres between sample wells. Journal of Negative Results in BioMedicine 6, 6 (2007).
13. Sakov, A. & Bickel, P. J. An Edgeworth expansion for the m out of n bootstrapped median. Statistics & Probability Letters 49, 217-223 (2000).

What is claimed is:

1. A method for analyzing quantities of one or more analytes between samples in an assay, the method comprising:
receiving data representing signals from detectably-labeled reporters bound to analytes which are bound to beads through a binding member specific for an analyte of interest and an embedded code, wherein the data includes replicate values for each analyte of interest;
adjusting for differences in replicate values to produce adjusted replicate values;
combining the adjusted replicate values to produce combined data;
applying a transform to the combined data to produce transformed data, wherein the transformed data exhibits a stabilized variance; and
comparing the transformed data to transformed data from at least one reference condition to determine a significance of differences between conditions.

2. The method of claim 1, wherein the received data is from a Luminex assay.

3. The method of claim 1, wherein the beads are xMAP® beads.

4. The method of claim 1, wherein the transform is a logarithmic transform.

5. The method of claim 1, wherein the transform is an inverse transform.

6. The method of claim 1, wherein the transform is of the form xⁿ, where n is not equal to 1.

7. The method of claim 1, wherein the comparison is performed using a bioequivalence-type test that compares treatment samples to control samples.

8. The method of claim 7, wherein the bioequivalence-type test includes an equivalence margin that is determined by a power estimation on the received data.

9. The method of claim 1, wherein the reporter is an antibody that selectively binds to an analyte of interest.

10. The method of claim 1, wherein the analyte of interest is a cytokine.

11. The method of claim 1, further comprising estimating a treatment effect using a least squares algorithm.

12. A computer-readable medium including instructions that, when executed by a processing unit, cause the processing unit to analyze analyte concentrations between samples in a multiplex assay, by performing the steps of:
receiving data representing signals from detectably-labeled reporters bound to analytes which are bound to beads through a binding member specific for an analyte of interest and an embedded code, wherein the data includes replicate values for each analyte of interest;
adjusting for differences in replicate values to produce adjusted replicate values;
combining the adjusted replicate values to produce combined data;
applying a transform to the combined data to produce transformed data, wherein the transformed data exhibits a stabilized variance; and
comparing the transformed data to transformed data from at least one reference condition to determine a significance of differences between conditions.

13. The method of claim 12, wherein the received data is from a Luminex assay.

14. The method of claim 12, wherein the beads are xMAP® beads.

15. The method of claim 12, wherein the transform is a logarithmic transform.

16. The method of claim 12, wherein the transform is an inverse transform.

17. The method of claim 12, wherein the transform is of the form xⁿ, where n is not equal to 1.

18. The method of claim 12, wherein the comparison is performed using a bioequivalence-type test that compares treatment samples to control samples.

19. The method of claim 18, wherein the bioequivalence-type test includes an equivalence margin that is determined by a power estimation on the received data.

20. The method of claim 12, wherein the reporter is an antibody that selectively binds to an analyte of interest.

21. The method of claim 12, wherein the analyte of interest is a cytokine.

22. The method of claim 12, further comprising estimating a treatment effect using a least squares algorithm.

23. A computing device comprising:
a data bus;
a memory unit coupled to the data bus;
a processing unit coupled to the data bus and configured to
receiving data representing signals from detectably-labeled reporters bound to analytes which are bound to beads through a binding member specific for an analyte of interest and an embedded code, wherein the data includes replicate values for each analyte of interest;
adjusting for differences in replicate values to produce adjusted replicate values;
combining the adjusted replicate values to produce combined data;
applying a transform to the combined data to produce transformed data, wherein the transformed data exhibits a stabilized variance; and
comparing the transformed data to transformed data from at least one reference condition to determine a significance of differences between conditions.

24. The computing device of claim 23, wherein the memory includes instructions that, when executed by the processing unit, cause the processing unit to receive the data, adjust for differences, combine the adjusted replicate values, apply the transform, and compare the transformed data.
